(19) United States
(12) Patent Application Publication
(10) Pub. No.: US 2003/0050704 A1
(43) Pub. Date: Mar. 13, 2003

(54) Prosthesis

(76) Inventor: Ory Keynan, Givataim (IL)

Correspondence Address: FROMMER LAWRENCE & HAUG, 745 FIFTH AVENUE- 10TH FL., NEW YORK, NY, 10151, US

(21) Appl. No.: 10/188,122

(22) Filed: Jul. 2, 2002

Related U.S. Application Data
(63) Continuation of application No. PCT/IL01/00001, filed on Jan. 1, 2001.

(30) Foreign Application Priority Data
Jan. 1, 2001 (IL) 133873
Jan. 3, 2000 (IL) 133874

(51) Int. Cl. A61F 2/32

(52) U.S. Cl. 623/22.12; 623/22.4

(57) ABSTRACT

A prosthetic device for an enarthrosis of an extremity, such as the hip joint of the shoulder joint of a human body. The prosthetic device has a ball prosthesis portion and an optional socket prosthesis portion. The ball prosthesis portion is non-inflatable, having a ball member joined to an axial end of a body member via a neck member, and is securable to the extremity. In particular, the ball prosthesis portion has a transverse profile that is adapted for enabling the same to be implanted in the enarthrosis via a suitable portal formed through the lateral cortex of the long bone of the enarthrosis.

### TECHNICAL FIELD

The present invention relates to a prosthetic device and system for replacing the hip or shoulder joint, in particular such a device and system that requires reduced surgical intervention, and a surgical procedure for implanting such a prosthetic device/system in a shoulder or hip joint.

### BACKGROUND

Ball and socket joints in the human body include the hip joint and the shoulder joint. The hip joint is a ball and socket joint with six degrees of freedom allowing for movement towards, flexion, extension, abduction, adduction, internal rotation and external rotation, and comprises a femur received in the acetabulum for enabling rotational movement with respect thereto. The femur head is joined via the neck to the upper portion of the femur, comprising the greater and lesser trochanter in the neck region, with the intertrochanteric region being the line that passes between the two trochanters. The femoral canal, or medulla, extends from the proximal part to the distal part of the femur in line with femoral shaft. The angle between the neck and shaft of the femur varies between about 120°-150°, averaging about 125° in the elderly. The healthy femoral head is lined with articular cartilage which allows for smooth articulation between the femur and acetabulum, which is a cup-shaped bony structure which is continuous with the pelvis and lined with articular cartilage The two major pathologies about the hip joint that require surgery are trauma and osteoaritis. Traumatic fractures about the hip joint can involve the femur, the acetabulum, or both. One subgroup of femoral fractures involves fractures that are currently generally treated by surgical replacement of the femoral head with a metal prosthesis. Proximal femoral fractures are usually divided into intra-articular fractures or subcapital fractures, where the fracture line goes through the femoral neck within the joint capsule, and extra-articular or pertrochanteric fractures, where the fracture line goes through the intertrochanteric region, outside the joint capsule. Subcapital fractures are more problematic than pertrochanteric fractures because a subcapital fracture often compromises the blood supply to the femoral head, which can lead to the head's consequent necrosis. It is for this reason that pertrochanteric fractures are always reduced and fixed, whereas subcapital fractures are reduced and fixed only in special cases (undisplaced or minimally displaced fractures and/or fractures in young patients), while in other cases it is assumed that the damage caused to the blood supply during the trauma will eventually lead to the necrosis of the femoral head, and the femoral head is therefore surgically removed and replaced with a metal prosthesis in a procedure known as hip hemiarthroplasty.

Osteoarthritis is a degenerative process whereby the hip joint loses the cartilaginous covering of the femoral head and/or acetabulum, leading to decreased range of motion and pain in the hip joint, and is usually treated in its most severe form by replacement of both the femoral head and the acetabulum This procedure is called total hip arthroplasty.

Current accepted surgical treatment for femoral fractures include internal fixation or hip hemiarthroplasty, while the most common surgical intervention performed for advanced symptomatic osteoarthritis include total hip arthoplasty. In hip hemiarthroplasty the current technique involves incision of the skin, different muscle groups (depending on the specific approach used), and the hip capsule. Then the femoral head is removed via an osteotomy (i.e., it is sawed off, with part of the neck about a centimeter above the lesser trochanter), and the hip joint dislocated. Next, the femoral canal is prepared using bone reamers in the shape of the intended prosthesis. Then, a metal alloy prosthesis is inserted into the medullary canal with a head size matching the original femoral head size, and the hip joint is manually reduced. Finally the soft tissue envelope is sutured. There are different types of prostheses, which entail slight changes in the surgical technique.

In total hip arthroplasty, current widely-used techniques involve incision of the skin, different muscle groups (depending on the specific approach used), and excision of the hip capsule. Then, the femoral head is removed with part of the neck, after an osteotomy is made in the femoral neck. Next the acetabulum is prepared for implantation of a polyethylene cup, by reaming the remains of the articular cartilage and part of the underlying bone. Then the cup is implanted either with or without cement, depending on the decision made before surgery by the surgeon. Next, the femoral canal is prepared as described for hemiarthroplasty, and a metal alloy femoral prosthesis is inserted with or without cement. Finally, the joint is manually reduced and part of the soft tissue envelope is sutured (usually the hip capsule is sacrificed and most of the muscles incised are not sutured except for their outer envelope, i.e. fascia). Again, there are many types of prostheses for the femur and acetabulum, which entail minor variations in the above procedure.

The shoulder girdle of a human consists of three bones: the humerus, the scapula, and the clavicle. The humerus consists of a hemispherical head, joined to the humeral shaft via a neck region comprising the greater and lesser tuberosity with the biccipital groove being the line that passes between the two tuberosities, serving as a trochlea for the tendon of the biceps muscle. The humeral canal, or medulla, extends from the proximal part to the distal part of the humerus in line with humeral shaft. The angle between the neck and shaft of the humerus varies between 130°-140°. The healthy humeral head is lined with articular cartilage which allows for smooth articulation between the humerus and scapular glenoid

The scapula glenoid is a pear-shaped concave bony structure having a short neck and lined with articular cartilage, reinforced on its outer rim by a fibrous structure called the labrum. Together with the humeral head, the glenoid forms the glenohumeral joint. The glenohumeral joint is a shallow ball and socket joint with six degrees of freedom allowing for movement towards flexion, extension, abduction, adduction, internal rotation and external rotation.

The two major pathologies of the shoulder which require arthroplasty are trauma and osteoarthritis. Fractures about the glenohumeral joint can involve the humerus, the glenoid, or both, and a special subgroup of the humeral head fractures is currently generally treated by surgical replacement of the humeral head with a metal prosthesis. Proximal humeral fractures are classified according to the number and type of fracture fragments. The four basic fragments referred to are: the articular portion of the humeral head (the fracture line goes through the anatomical neck), the shaft portion, or subcapital fractures (the fracture line goes through the surgical neck), the greater tuberosity, and the lesser tuberosity. If only one of the above fragments is involved it is considered a two-part proximal humeral fracture, whereas when all the above fractures coexist, it is called a four-part fracture. Replacement of the humeral head by prosthesis (hemiarthroplasty) is usually the treatment of choice for four-part fractures and/or fracture lines which split the humeral head up to its articular cartilage.

Osteoarthritis is a degenerative process whereby the glenohumeral joint loses the cartilaginous covering of the humeral head and/or glenoid, leading to decreased range of motion and pain in the joint. It is usually treated in its most severe form by replacement of both the humeral head and the glenoid. This procedure is called total shoulder arthroplasty.

Similarly to the case with the hip joint, current surgical treatment for these conditions include shoulder hemiarthroplasty and total shoulder arthroplasty, both techniques involving incision of the skin, of different muscle groups, and of the joint capsule.

In shoulder hemiarthroplasty, after incision of the skin, different muscle groups, and of the joint capsule, the humeral head is removed via an osteotomy (i.e., it is sawed off, with part of the neck), and the glenohumeral joint dislocated. Next, the humeral canal is prepared using bone reamers in the shape of the intended prosthesis. Then, the metal alloy prosthesis is inserted into the canal with a head size roughly matching the original humeral head size, and the joint is manually reduced. Finally the soft tissue envelope is sutured. There are different types of prostheses which entail slight changes in the surgical technique.

In total shoulder arthroplasty, after incision of the skin, different muscle groups, and joint capsule, the humeral head is removed with part of the neck, after an osteotomy is made in the anatomical neck. Next the glenoid is prepared for implantation of a polyethylene cup, by reaming the remains of the articular cartilage and part of the underlying bone. Then the cup is implanted with cement or with a metal backing outer shell. Next, the humeral canal is prepared as described for hermiarthroplasty, and a metal alloy humeral prosthesis is inserted with or without cement. Finally, the joint is manually reduced and the soft tissue envelope and the detached bony fragments (tuberosities) are sutured. Again, there are different types of prostheses for the humerus and glenoid, which entail minor variations in the above procedure. Regardless of the specific surgical approach used, both these procedures—Hemiarthroplasty and Arthroplasty—involve extensive dissection and disturbance of the normal anatomy of the joint in question—i.e., whether the hip joint or of the shoulder joint—usually involving the abductors and internal rotators, after which only part of the soft tissue envelope of the joint is restored. This diminishes the contribution to the stability of the joint afforded by the joint capsule and surrounding muscles and increasing the risk of dislocation of the prosthesis. In addition, the strength of the muscles necessary for postoperative rehabilitation is diminished, thus necessitating longer postoperative rehabilitation and hospitalization. Furthermore, the wide exposure of the hip joint necessary for currently used technique exposes the joint to risk of infection. Also, the use of a metallic prosthetic head to articulate with bone, as is done with hemiarthroplasty, causes erosion of the acetabulum or of the glenoid, depending on the particular joint, which often leads to post operative pain and consequent conversion to total hip arthroplasty or total shoulder arthroplasty, respectively. The surgical scar is prominent and cosmetically unacceptable. Particularly in the case of the hip joint but also in the shoulder joint, the stress shielding of current femoral prosthesis design and of the humeral prosthesis design, respectively, is a major contributing factor to prosthesis loosening, for which no solution has been found to date.

Clearly, there are many areas of improvement to these procedures, both in the case of the hip joint and the shoulder joint, as well as in the configuration of the prosthesis used. For example, maintaining the integrity of the soft tissue envelope of the joint would result in improved postoperative stability of the joint and would decrease the postoperative incidence of dislocation, as well as shortening rehabilitation and hospitalization time. Limiting the surgical exposure would lower the incidence of postoperative infection. Lowering the coefficient of friction found in hemiarthroplasty with the currently used metal-on-bone solution, and lowering the stress shielding on bone caused by currently used femoral stem designs and humeral stem designs, would each be beneficial to the patient. In some cases leaving a smaller, more cosmetically acceptable scar would be a significant advantage.

In U.S. Pat. No. 4,714,478 a flexible hollow collapsible prosthesis is described together with a surgical procedure for its installation via a portal made through the femoral cortex opposite the head of the femur. Insertion of the prosthesis is carried out in the collapsed condition, and when in place it is filled in via an access opening, considerably increasing its displacement volume. While this procedure reduces trauma to the patient to some extent in relation to other prior art methods, there are still a number of disadvantages to this procedure. Most notably, the lack of anchoring of the prosthesis in the femoral shaft renders the prosthesis vulnerable to shear stresses during weight bearing conditions. Further, the material from which the prosthesis is made is less suitable than traditional metals being weaker and less wear-resistant, and the ability of the material to bear the weight of the patient for long periods is questionable. In addition, the use silicone is frequently responsible for chronic inflammation in surrounding tissue when used in vivo, and it is therefore not particularly suitable in the current context. Also, the possibility exists of the prosthetic shell puncturing and leaking the contents thereof into the joint space causing potential mechanical problems in the joint and/or causing infection.

It is therefore an aim of the present invention to provide a prosthetic device and system, and method of implantation, which overcomes the limitations of prior art prosthetic devices and methods of implantation thereof.

It is another aim of the present invention to provide a prosthetic device and system for the hip joint such as to require substantially less surgical intervention for its implantation than current devices.

It is another aim of the present invention to provide a prosthetic device and system for the shoulder joint such as to require substantially less surgical intervention for its implantation than current devices.

It is another aim of the present invention to provide such a prosthetic device and system, and procedure for its implantation, for maintaining the integrity of the soft tissue envelope of the joint.

It is another aim of the present invention to provide such a prosthetic device and system, and procedure for its implantation, that limits the surgical exposure of the joint.

It is another aim of the present invention to provide a prosthetic device and system such as to lower the coefficient of friction found in hemiarthroplasty relative to the currently used metal-on-bone solution, and to lower the stress shielding on bone caused by currently used femoral stem designs.

It is another aim of the present invention to provide a non-collapsible prosthetic device and system, and procedure for its implantation, that results in a smaller, more cosmetically acceptable scar.

### SUMMARY OF INVENTION

The present invention relates to a prosthetic device for an enarthrosis of an extremity, such as of the hip joint or of the shoulder joint of a human body, comprising

a substantially non-collapsible or non-inflatable ball prosthesis portion comprising a ball member joined to an axial end of a body member via a neck member;

wherein said ball prosthesis portion comprises a transverse profile adapted for enabling at least said ball prosthesis portion to be implanted in said enarthrosis via a suitable portal formed through the lateral cortex of the long bone of the extremity of the enarthrosis.

Said body member is preferably substantially cylindrical and is optionally adapted for securement thereof within said portal. The transverse profile of said body member is typically substantially complementary to the transverse profile of said portal. The ball member preferably comprises an equatorial diameter similar to a transverse diameter of said body member.

The prosthetic device optionally further comprises a socket prosthesis portion having a proximal end adapted for securement onto the socket of said enarthrosis and a distal side having a concavity adapted for receiving said ball member such as to permit relative rotational movement therebetween. The concavity of said socket prosthesis portion preferably has a curvature complementary to the curvature of said ball portion.

The ball prosthesis portion is typically substantially rigid and may be made from a metallic material including stainless steel; titanium, titanium alloys, and cobalt-chromium-molybdenum alloys or from a ceramics material including zirconium oxide. The socket prosthesis portion may be made from silicone, polyethylene, polyurethane, ceramic or any other suitable material

The prosthetic device optionally further comprises securement means for anchoring said ball prosthesis portion with respect to an extremity. The securement means may comprise a suitable compression screw arrangement comprising a side plate joined to a distal end of said body portion, and a compression screw adapted for bringing said side plate into abutting contact with the corresponding long bone of said enarthrosis, and further comprising at least one nail for securing said side plate onto said long bone. Alternatively, the securement means may comprise a suitable intramedullary nail arrangement comprising an intramedullary nail adapted for securement thereof within a shaft formed in a medullary canal, and mounting means for reversibly mounting said ball prosthesis portion to said intramedullary nail. Alternatively, the securement means may comprise a suitable telescopic strut arrangement comprising an extendable telescopic strut adapted for securement thereof within a shaft formed in a medullary canal, and mounting means for reversibly mounting said ball prosthesis portion to said telescopic strut arrangement. In the latter case, the telescopic strut preferably comprises in its retracted configuration a transverse profile adapted for enabling said ball prosthesis to be implanted in said enarthrosis via said distal portal. Optionally, the telescopic strut comprises an outermost member integrally joined to said distal end of said body member.

The prosthetic device may be adapted for the hip joint or for the shoulder joint.

The present invention also relates to a method for installing a prosthetic device for the hip joint or the shoulder joint

### DESCRIPTION OF FIGURES

FIG. 1 shows in side view part of the anatomy of a hip joint.

FIG. 2 shows in side elevational cross-sectional view, a typical prior art prosthesis used for the hip joint.

FIG. 3(a) shows in side elevational cross-sectional view, a preferred embodiment of the prosthetic device of the present invention adapted for use in the hip joint, and FIG. 3(b) shows the ball/cup arrangement in greater detail.

FIG. 4 shows the embodiment of FIG. 3 comprising a side plate securement means.

FIG. 5 shows the embodiment of FIG. 3 comprising an intramedullary nail/strut securement means.

FIG. 6 shows the embodiment of FIG. 3 comprising a telescopic strut securement means.

### DISCLOSURE OF INVENTION

The present invention is defined by the claims, the contents of which are to be read as included within the disclosure of the specification, and will now be described by way of example with reference to the accompanying Figures.

In the present specification, the term “distal” refers to a direction away from the trunk or body of the patient, while the term “proximal” refers to a direction towards the trunk or body of the patient.

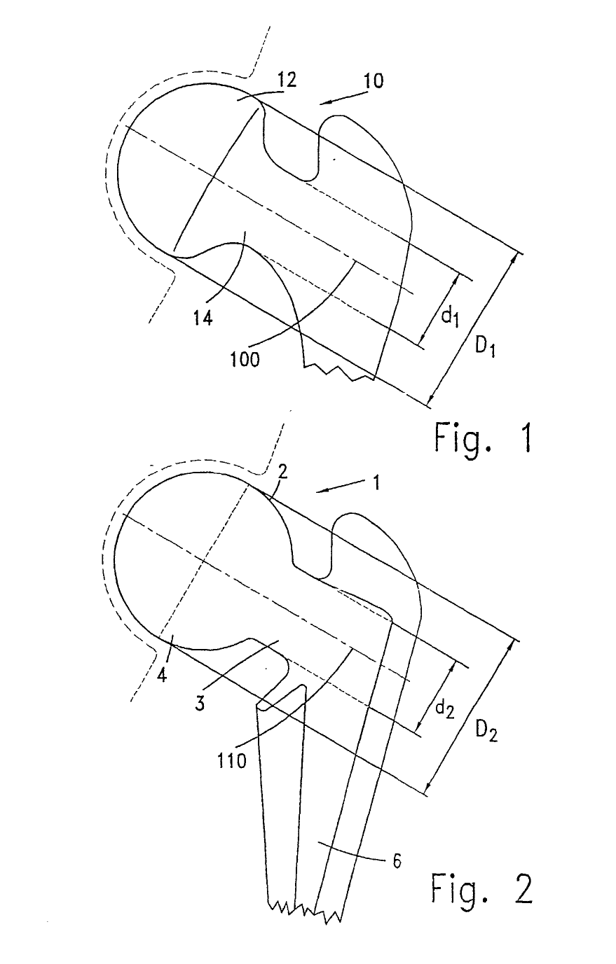

According to the current state of the art, a prosthetic device for replacing solely the ball portion of a ball-and-socket joint (i.e., the hip joint or the shoulder joint of a human body), as in hemiarthroplasty, comprises an enlarged ball portion of similar proportions to the ball or head of the joint that it is to replace. Thus, referring to FIGS. 1 and 2, the transverse diameter (D2) at the equatorial portion (2) of a head (4) of a typical prior art prosthesis (1), typically perpendicular to the axis (110) of the neck portion (3), is substantially similar to the corresponding diameter (D1) of the head (12) of a femur (10) that the prosthesis (1) is to replace, and significantly larger than the diameter (d1) of the neck portion (14) of the femur (10) itself. The prior art prosthesis (1) also typically comprises a neck portion (3) and a stem portion (6) that is typically anchored in the medullary canal. The axis (100) of the neck (14) of the femur or humerus may be defined as an imaginary line from the centre of the head running through the midline of the neck and extending to a point about midway between the greater and lesser trochanters. The axis (100) is typically the rotational axis of the long bone such as the femur (10) with respect to the joint. The central axis (110) of the neck (3) of the prior art prosthesis (1) is substantially aligned with the axis (100) of the neck (14) of the femur (10) that the prosthesis (1) is replacing. This configuration for the prior art prosthesis (1) provides compatibility with the existing dimensions of socket portion of the ball and socket joint, or enarthrosis. Nonetheless, the relatively large size of the prosthesis head (4) substantially determines that the surgical procedure for implantation/insertion of the prosthesis requires an extensive surgical approach entailing substantial trauma to the soft tissue envelope of the joint, with the ensuing problems and disadvantages discussed above. The situation regarding the shoulder joint is similar to that described above for the hip joint, mutatis mutandis.

The present invention is directed to an improved prosthetic device for replacing at least the ball portion of a ball-and-socket joint, or enarthrosis, of a limb or extremity, such as of the hip joint or of the shoulder joint of a human body. The present invention is thus directed to an improved prosthetic device comprising:—

a substantially non-collapsible or non-inflatable ball prosthesis portion comprising a ball member joined to an axial end of a body member via a neck member;

wherein said ball prosthesis portion comprises a transverse profile enabling said ball prosthesis to be implanted in said enarthrosis via a suitable portal formed through the lateral cortex of the long bone of the enarthrosis, typically in the proximal part of the long bone.

Such a system preferably further comprises a socket prosthesis portion having a proximal end adapted for securement onto the socket of said enarthrosis and a distal side having a concavity adapted for receiving said ball member such as to permit relative rotational movement therebetween.

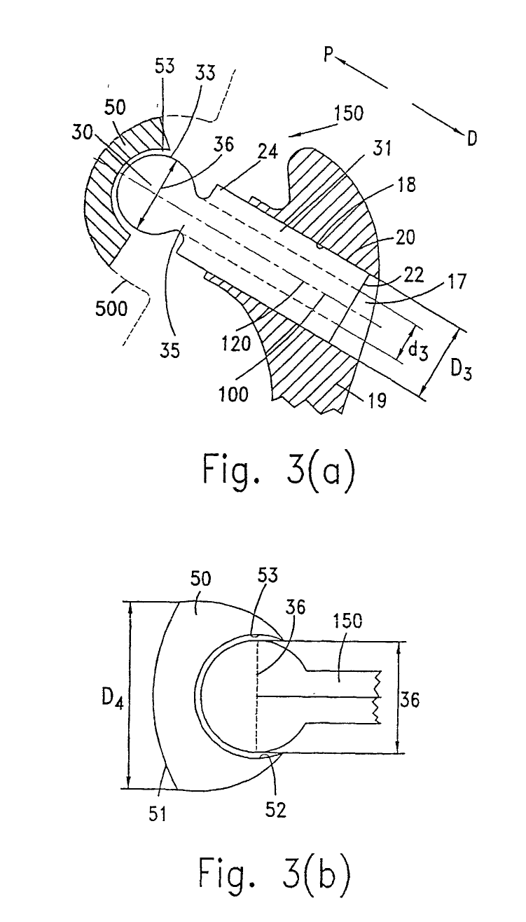

Referring to FIGS. 3(a) and 3(b), the ball prosthesis, generally designated (150), in particular the ball member (30) thereof, is non-collapsible or non-inflatable, and substantially non-flexible, and is thus designed to substantially maintain its shape and size before, during and after installation, and during use. The ball prosthesis (150) comprises a body member (31) adapted to be accommodated in a portal (17) formed in the long bone (i.e., the femur or humerus) of the extremity or limb, and thus in the preferred embodiment comprises a substantially cylindrical member (20) having distal and proximal ends, (22) and (24) respectively. Alternatively, said body member (31) may comprise any other suitable cross-sectional shape, including, for example, substantially rectangular, and is preferably of constant section along the longitudinal axis (110) thereof. A ball member (30) is joined to a neck member (35) on said proximal end (24), the neck member being substantially narrower than said ball member (30).

The socket prosthesis (50) has a proximal end (51) adapted for securement onto the socket of the enarthrosis, and a distal side (52) having a concavity (53) adapted for receiving said ball member (30) such as to permit relative rotational movement therebetween. Said differently, the said ball member (30) has a substantially rounded surface (33) adapted for fitting in a complementary cup-like cavity (53) of a socket prosthesis (50) such as to permit movement in substantially any direction with respect to said cavity (53). If the ball prosthesis (150) is for a hip joint, then the socket prosthesis (50) is adapted to fit and be secured in the acetabulum (500) of the particular patient. Optionally, such a socket prosthesis for the hip joint may made from silicone, polyethylene, polyurethane, ceramic or any other suitable material. Typically, the socket prosthesis is in the form of a cup-shaped component made from high molecular weight polyethylene (HMWPE), the inner curvature of the cup being substantially complementary to the curvature of the said ball member (30), and the outer curvature of the cup being substantially complementary to the curvature of the hip socket. Such a cup-shaped component may further comprise a series of grooves on the outer surface, preferably in the form of concentric circles radiating from the convex apex of the cup, for providing anchoring points for bone cement. Alternatively, if the prosthesis (150) is for the shoulder joint, then the socket prosthesis (50) is adapted to fit and be secured in the glenoid, of the particular patient. Typically such a socket resurfacing prosthesis for the shoulder joint may comprise, for example, a prosthesis similar to a “Neer II Resurfacing Glenoid Prosthesis”, known in the art, which is essentially a shallow polyethylene cup roughly the size of a human glenoid, but having an inner curvature substantially complementary to the curvature of the prosthetic ball member (30), which is smaller than corresponding prior art prosthetic heads (4). Such a glenoid prosthesis may further comprise a fin in its non-articulating side for engaging the glenoid. The procedure by which the socket prosthesis (50) is placed and secured in the particular bone socket is discussed below.

The prosthesis device of the present invention is characterised in that the ball prosthesis (150) comprises a transverse profile enabling said ball prosthesis (150) to be implanted in said enarthrosis via a suitable portal (17) formed through the lateral cortex (19) of the proximal part of the limb. By transverse profile of the ball prosthesis (150) it is meant the size and/or shape of the various portions of the ball prosthesis (150), in particular of the ball member (30) and of the body member (31), in planes normal to the longitudinal axis (120) of the ball prosthesis (150)—and therefore to the axis of the portal (17). Thus, both the shape and size of at least the ball member (30) and of at least a proximal portion of the body member (31) is such as to enable these portions to navigate through the portal (17) into position in the joint capsule—in other words to enable at least these parts of the ball prosthesis (150) to be inserted into the joint capsule via the portal (17). In its simplest form, then, the transverse diameters of the ball member (30) and of the cylindrical member (20) are thus smaller, or at least not greater than, the diameter of the portal (17), such as to enable the ball prosthesis (150) to be inserted into the hip or shoulder capsule from a lateral direction via a portal (17) in the femur or humerus, respectively, the portal (17) being specially bored into the corresponding femur or humerus substantially co-axially with axis (100) of the neck section (14) thereof. This procedure is discussed below. In the femur, the maximum diameter (D3) of the portal (17) is limited by the diameter of the neck (14) of the femur, and/or the amount of bony tissue left in the greater and lesser trochanters such as to provide sufficient mechanical integrity of the upper part of the bone. Thus, at least the ball member (30) of the prosthesis (150) comprises an equatorial diameter (36), taken as substantially perpendicular to the central axis (120) of the prosthesis (150), not greater than said maximum diameter (D3). Such an equatorial diameter (36) is thus significantly smaller than the equatorial diameter of the original bony head (12) that the prosthesis (150) is replacing, and is typically of the same order as the diameter (d1) of the neck region (14) of the femur. The central axis (120) of the prosthesis (150) is substantially aligned with the axis (100) of the neck section (14) of the femur (10). Alternatively, the portal (17) may be substantially rectangular in transverse cross-section, and the body member (31) thus advantageously comprises a rectangular cross-sectional profile complementary thereto. In this case, the diameter of the ball member (30) is typically slightly smaller than the minimum width of the portal (17), for example.

The situation with respect to a prosthesis (150) adapted for the humerus is similar-to that described hereinbefore with respect to the femur, mutatis mutandis.

The neck (35) of the prosthesis (150) is sized in relation to the ball member (30) thereof such as to provide the required rotational movement between the socket prosthesis (50) and the prosthesis (150). Nonetheless, this needs to be balanced with providing a mechanically sound join between the ball member (30) and the body member (31) capable of withstanding the stresses applied thereto via the joint. In the preferred embodiment, the said cylindrical member (20) preferably comprises an external diameter close to the internal diameter of the portal (17), such as to provide a tight fit between the two when inserted therein. Alternatively, though, a small radial clearance may be provided between the said cylindrical member (20) and the portal (17) to enable a suitable adhesive, such as bone cement including polymethamethylacrylate (PMMA) for example, to be applied between the portal (17) and the member (20) when inserted therein to secure the latter in place. In such a case, the surface of the cylindrical member (20) may advantageously comprise one or more rivulets, preferably arranged circumferentially and/or spirally and/or axially. Alternatively, the diameter of the cylindrical member (20) my be significantly smaller than that of the portal (17), typically about the same as that of the neck (35), for example, the prosthesis (150) further comprising one or more suitable sleeves coaxial with the cylindrical member (20), and sized such as to enable the latter to be secured within the portal (17).

The said prosthesis (150) is preferably of integral construction, and made from any suitable bio-compatible material, preferably chosen from among: stainless steel, titanium, titanium alloys, “supermetal” alloys including cobalt-chromium-molybdenum, and ceramics including zirconium oxide.

The said prosthesis (150) may be unitary, in which at least the said ball member (30), neck member (35) and cylindrical member (20) are integrally joined. Alternatively, each of these components, i.e., at least the ball member (30), neck member (35) and cylindrical member (20), may comprise suitable means for releasably or permanently joining one to the other in series. For example, the ball member (30) may comprise a radial spike for press-fitting reversibly or non-reversibly into a suitable recess in the neck member (35). Similarly, the neck member (35) may comprise a suitable arrangement to enabling it to be press-fitted into the cylindrical member (20). In fact, each of these components may be further adapted to be modular, so that, for example, different sized ball members (30) may be interchangeably joined to any one of a range of neck members (35), which may be in turn optionally interchangeably joined to any one of a range of cylindrical members (20). Thus, the modularity of the various prosthesis components (as well as the choice of material which constitutes the prosthesis) leads to great flexibility in constructing prostheses suitable for a wide range of anatomical variations, and is also simplifies logistics of such items for hospitals and similar institutions.

Such a prosthesis (150) adapted for the glenohumoral joint is similar to that adapted for the hip joint as described above, mutatis mutandis, the main differences being dimensional, the differences in the dimensions of the prosthesis body, neck and head and angles between the above-mentioned components allowing for differences in the regional anatomy of the shoulder as compared to the hip.

The ball prosthesis (150) further comprises securement means, preferably at the distal end of the said cylindrical member (20), for mechanically fixing said prosthesis (10) in place with respect to the long bone, i.e., the femur or humerus. While the said cylindrical member (20) may optionally be cemented or press-fitted into said portal (17), such methods of securement are generally not sufficient to support the prosthesis (150) during dynamic movements of the limb or to fully support the weight of the patient, at least not for extended periods, and further securement is needed. According to the present invention, such securement means may comprise any suitable mean for securing the prosthesis (150) to the long bone of the limb, i.e., the corresponding femur (or humerus, mutatis mutandis).

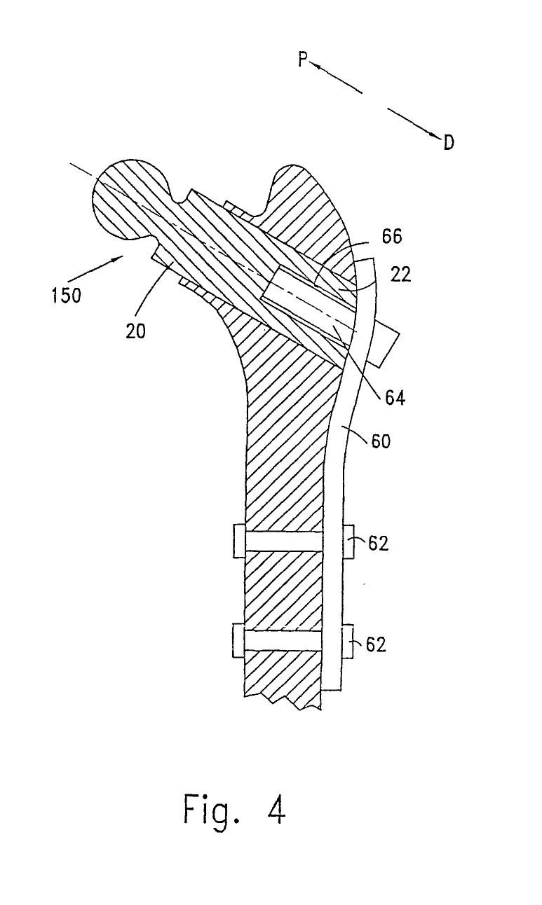

Referring to FIG. 4, in one embodiment of the present invention the securement means comprises a side plate (60) which is mounted onto the femur (or humerus, mutatis mutandis) via screws (62) in a manner known in the art. The side plate (60) secures the prosthesis (150) in place via a compression screw (64) which can be screwed into a complementary shaft (66) comprised in the distal end (22) of the cylindrical member (20), the shaft (66) and compression screw (64) comprising complementary screw-threaded surfaces. Optionally, the plate (60) may be integrally formed at least with the cylindrical member (20), or with the ball prosthesis (150).

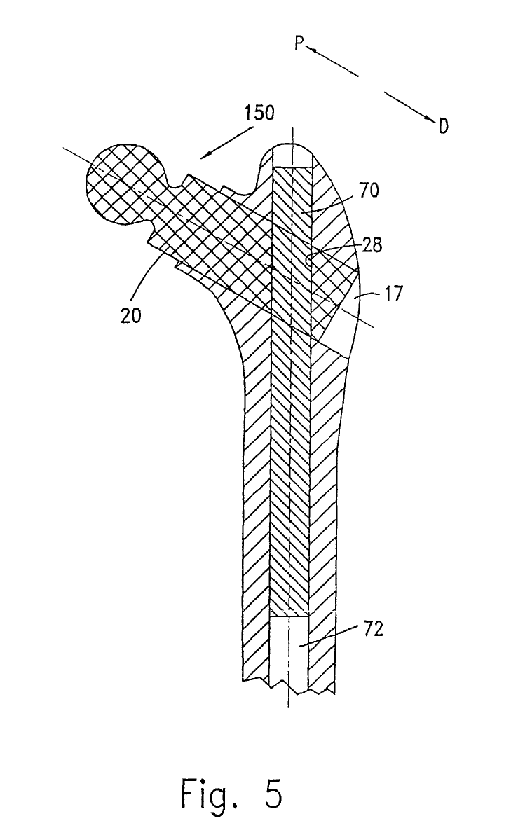

Alternatively, said securement means may comprise an intramedullary nail or strut (70), which may be mounted transversely onto said cylindrical member (20) at the required angle via transverse shaft (28) therein using any suitable means. The strut or nail (70) may be inserted into a specially prepared cavity (72) in the medullary canal of the femur or of the humerus, in the case of a hip joint or shoulder joint, respectively, as illustrated in FIG. 5. Procedures for preparing said cavity (72), for securing the nail (70) therein, and for mounting the nail (70) to a cylindrical member such as a compression hip screw or nail are known in the art. Such procedures may be adapted straightforwardly for the securement of said cylindrical member (20) in place of such a compression hip screw.

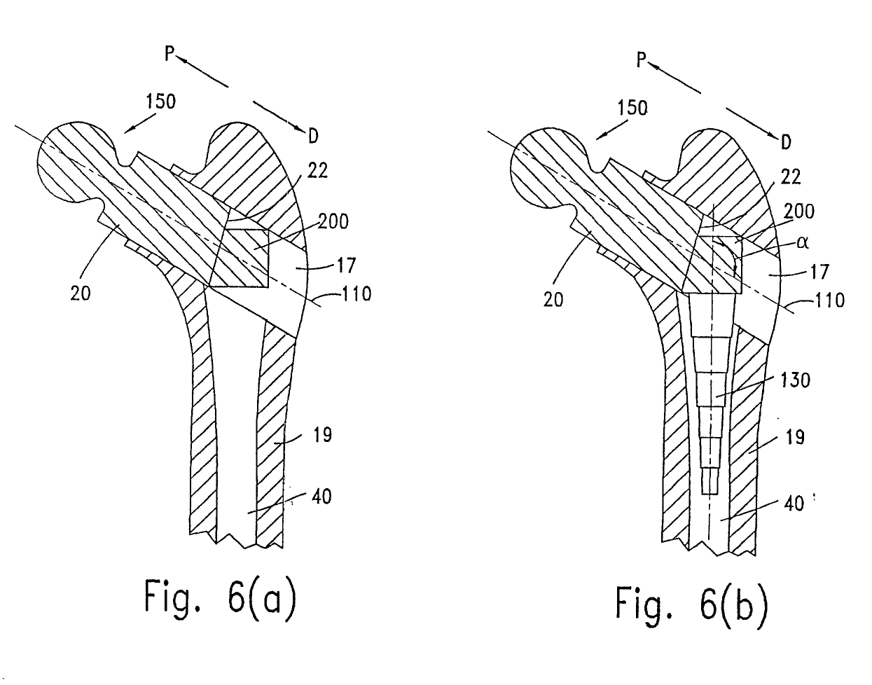

In the preferred embodiment, and referring to FIGS. 6(a) and 6(b), the said securement means comprises a suitable telescopic strut (200). Examples of such a suitable telescopic strut are disclosed in co-pending Israel Patent Application No. 133874 by applicant, entitled “Intramedullary Support Strut”, the contents of which are incorporated herein in their entirety.

As described in said co-pending application, the telescopic strut (200) is characterised in that in its retracted configuration, it comprises a profile such as to insertable within portal (17), said telescopic strut (200) being transversely extendible into, and securable within, a shaft (40) prepared in the medullary canal. The said telescopic strut (200) is removably or permanently attached to the distal end (22) of the cylindrical body (20), or indeed integral therewith, and comprises a transverse profile (in the retracted configuration) which is preferably enclosed or circumscribed by the transverse profile of the said body member (31). Thus, where the telescopic strut (200) comprises a substantially rectangular transverse cross-sectional profile in the retracted configuration, the portal (17) may be advantageously formed with a complementary rectangular cross-section as well, for example, or with a circular traverse cross-section that cicumscribes the transverse perimeter of the strut (200). The axial dimension of the cylindrical member (20) is also such as to ensure that when this is properly positioned within the portal (17) the telescopic strut (200) is aligned with said shaft (40) previously bored into the medullary canal of the femur (or of the humerus, mutatis mutandis). The central axis (130) of the telescopic strut (200) is an appropriate angle a to the axis of the cylindrical member (20), particularly when the telescopic strut is fully extended. The telescopic member may be extended into the shaft (40) and cemented or otherwise secured therein, thereby providing suitable anchoring for the ball prosthesis (150) with respect to the femur (or humerus, mutatis mutandis).

The surgical procedure for implanting the said prosthesis (150) in the hip joint of a patient comprises the following steps:—

(a) Forming a Portal (17) Through the Lateral Cortex of the Femur Opposite the Head of the Femur and Removing the Head of the Femur.

First, an approximately 3 cm. skin incision is made in the lateral thigh centered with respect to the greater trochanter of the femur. Then, a similar incision is made in the fascia lata, and the Vastus Lateralis Muscle is split longitudinally along its fibers and depthwise until reaching the greater trochanter, the slit being of a similar length as the fascia lata incision. These incisions and split are retracted with a special automatic or alternatively with a manual retractor, and such retractors are well-known in the art. Then, a guide pin, typically a long metal pin having a sharp threaded tip, is inserted into the bone, centralised within the femoral neck and head (in both anteriorposterior (AP) and axial views), the insertion being typically carried out under fluoroscopy. A portal (17) is then formed extending from the side of the femur and through the neck portion (14) thereof. This may be achieved, for example, by removing a cylindrical section of bone from the femoral neck and head, centered over the guide, with a canullated cylindrical cutter. Alternatively, the portal (17) may be formed by drilling into the femur with any suitable drill, in particular such a drill adapted for being centralised on, and guided by, the guide pin. The femur head (12) is then removed. This may be performed in any number of ways. For example, a transverse cervical osteotomy of the femur is first made with any suitable device including a small diameter oscillating saw, for example, and the remains of the femoral head removed with a specially designed instrument or currently used bone reamers or burrs. Alternatively, the remains of the femoral head may be cut into transverse slices using specially adapted bladed tools or laser cutters or the like, and the sections removed via the portal with the aid of forceps, for example. Alternatively, the head of the femur may be removed using a special tool such as described in U.S. Pat. No. 4,714,478, for example, the contents of which are incorporated herein by reference thereto.

(b) Preparing the Acetabulum to Receive a Socket Prosthesis and Fixing Same in Place.

The acetabulum may be prepared typically by reaming with any suitable reamer such as a Charnley Acetabulum Reamer, for example, and optionally drilling several small holes in its articular surface for providing anchor points for the bone cement. The socket prosthesis (50) is then set and fixed in place in the acetabulum, using special bone cement such as polymethylmethacrylate (PMMA), for example. This may be accomplished in any number of ways. For example, the socket prosthesis (50) may comprise a HMWPE acetabulum cup having an inner curvature to match the ball member (30), this being inserted into the joint through the lateral hip incision made in step (a), with the open side (i.e. concavity (53)) facing the femur (face down) and an interval or space between the bone and overlying muscles is developed (with retraction) until the hip capsule is reached, incised longitudinally (along the axis (100) of the femoral neck (14)) and the socket prosthesis (50) is dropped into the joint. Then cement is injected and the cup is pressed (with a long-armed tool having a ball-shaped end—similar or smaller than said ball member (30)—inserted through the bony tunnel) against the acetabulum until the cement cures (typically about 12 minutes). Alternatively, the cup may be directly inserted into the joint through a separate ski incision in the groin, and fixed in place as before.

(c) Preparing Infrastructure in the Femur for the Securement Means of the Ball Prosthesis.

A number of different alternatives are possible for fixing and securing the ball prosthesis (150) within the portal (17) and in engagement with the socket prosthesis (50), depending on the particular securement means of the ball prosthesis (150). In the preferred embodiment, the ball prosthesis (150) comprises a telescopic strut (200) at its distal end (22). In this configuration, the required infrastructure in the femur is a shaft (40) prepared in the medullary canal. This may be accomplished with a minimally invasive procedure making use of the portal already formed in step (a) of the procedure. Essentially, a flexible guide is introduced distally from the intertrochanteric region into the medullary canal, followed by a flexible reamer threaded on the guide for reaming the proximal medullary canal. Alternatively, the shaft (40) may extend from the upper portion of the femur through the medullary canal, and such an extended shaft may be prepared by normal methods used for preparing a medullary shaft for an intramedullary nail.

Alternatively, for embodiments in which the securement means comprises an intramedullary nail or strut (70), a shaft (72) is made through the medullary canal in the normal way, beginning from the upper part of the femur. Alternatively, for embodiments in which the securement means comprises a side plate (60) and compression screw (64), the said infrastructure is a plurality of transverse apertures in the femur for inserting and locking therein a plurality of screws (62). However, with this type of securement means, this step is preferably performed after the ball prosthesis (150) is implanted in the joint.

(d) Insertion of the Ball Prosthesis Through the Portal.

The ball prosthesis (150) may then be inserted into the joint via the portal (17), until the ball member (30) is engaged in the concave or cup-like cavity (53) of the socket prosthesis (50). This may be done manually by pushing with fingers or alternatively with a special tool. Preferably, the ball prosthesis (150) is fixed in place within the portal (17) by cementing at least a portion of the cylindrical member (20) within the said portal (17). The cement is typically applied with the use of a suitable cement gun or applicator.

(e) Securing Said Ball Prosthesis (150) with Respect to the Femur via Securement Means.

This step will depend on the specific securing means used with the ball prosthesis (150), and in general with the infrastructure prepared in step (c) above. Thus, in the preferred embodiment, the ball prosthesis (150) comprises a telescopic strut (200) at its distal end. The telescopic strut (200) is positioned at the distal end (22) of the ball prosthesis (150) such that when the latter is fully inserted through the portal (17) and into engaging contact with the socket prosthesis (50), the telescopic strut (200) is properly aligned with the shaft (40) made in the medullary canal. The telescopic strut (200) is then opened and locked according to its particular opening mechanism, thereby extending into the shaft (40) and providing a suitable anchor for the ball prosthesis (150).

Alternatively, the telescopic strut (200) may be inserted first into the medullary canal such that it clears the portal (17). Then, the ball prosthesis (150) is inserted into the portal (17) into engaging contact with the socket prosthesis (50). An angled joining member (not shown) is joined (permanently, removably or integrally) to the distal end (22) of the ball prosthesis (150) such that when the latter is fully inserted through the portal (17) and into engaging contact with the socket prosthesis (50), the angled joining member is also properly aligned with the top of the strut (200) that is already accommodated in the shaft (40) made in the medullary canal. The angled joining member is then attached to the strut (200).

In embodiments comprising an intramedullary nail or strut (70) as the securement means, the ball prosthesis (150) is inserted into the portal (17) into engaging contact with the socket prosthesis (50). The angled aperture (28) is positioned at the distal end (22) of the ball prosthesis (150) such that when the latter is fully inserted through the portal (17) and into engaging contact with the socket prosthesis (50), the angled aperture (28) is properly aligned with the shaft (40) made in the medullary canal. The intramedullary nail (70) is then inserted into the medullary canal and through angled aperture (28) of the ball prosthesis (150) and secured with respect thereto by suitable means, thereby providing a suitable anchor for the ball prosthesis (150).

In embodiments in which the securement means comprises a side plate (60) and compression screw arrangement, the ball prosthesis (150) is inserted into the portal (17) into engaging contact with the socket prosthesis (50). The compression screw (64) is then screwed into the complementary shaft (66), bringing the plate (60) into abutting contact with the outer part of the femur. Then, unless already prepared in step (c), suitable holes are drilled into the femur, and side screws (62) are screwed through the plate (60) and into the femur in the normal way to provide a suitable anchor for the ball prosthesis (150).)

(f) Closure of Fascia, Subcutis and Skin.

This step will depend on the specific securing means used with the ball prosthesis (150), and in general with the infrastructure prepared in step (c) above and the securement procedure in step (e) above. In the preferred embodiment employing said telescopic strut (200), part of the bone cylinder previously removed when forming the portal (17) is replaced, so as to fill the distal part of the portal (17). However, if the portal (17) was formed by d g and no such bone cylinder is available, the distal part of the canal (17) is plugged by using bone graft from the removed femoral head. In embodiments comprising an intramedullary nail (70) as the securement means, similar methods may be used to plug the distal end of the portal (17).

Closure of the muscles and tissues around the hip joint is then accomplished in the normal way, according to the specific details thereof, and these procedures are well known.

The surgical procedure for implanting the ball prosthesis (150) adapted for the shoulder joint of a patient is similar to the procedure for the hip joint as hereinbefore described, mutatis mutandis, with the possible optional difference that the socket prosthesis (50) is not used, and therefore not implanted in step (a).

While in the foregoing description describes in detail only a few specific embodiments of the invention, it will be understood by those skilled in the art that the invention is not limited thereto and that other variations in form and details may be possible without departing from the scope and spirit of the invention herein disclosed.

1. A prosthetic device for an enarthrosis of an extremity, such as of the hip joint or of the shoulder joint of a human body, comprising
a substantially non-collapsible ball prosthesis portion comprising a ball member joined to an axial end of a body member via a neck member;
wherein said ball prosthesis portion comprises a transverse profile adapted for enabling at least said ball prosthesis portion to be implanted in said enarthrosis via a suitable portal formed through the lateral cortex of the long bone of said enarthrosis.

2. A prosthetic device as claimed in claim 1, wherein said body member is substantially cylindrical.

3. A prosthetic device as claimed in claim 1, wherein said body member is substantially rectangular.

4. A prosthetic device as claimed in claim 1, wherein said transverse profile of said body member is substantially complementary to the transverse profile of said portal.

5. A prosthetic device as claimed in claim 4, wherein said body member adapted for securement thereof within said portal.

6. A prosthetic device as claimed in claim 1, wherein said ball member comprises an equatorial diameter similar to a transverse diameter of said body member.

7. A prosthetic device as claimed in claim 1, wherein said ball prosthesis portion is substantially rigid.

8. A prosthetic device as claimed in claim 1, wherein said ball prosthesis portion is made from a metallic material including stainless steel, titanium, titanium alloys, and cobalt-chromium-molybdenum alloys.

9. A prosthetic device as claimed in claim 1, wherein said ball prosthesis portion is made from a ceramics material including zirconium oxide.

10. A prosthetic device as claimed claim 1, further comprising securement means for anchoring said ball prosthesis portion with respect to an extremity.

11. A prosthetic device as claimed in claim 10, wherein said securement means comprises a suitable compression screw arrangement comprising a side plate joined to a distal end of said body member, and a compression screw adapted for bringing said side plate into abutting contact with the corresponding long bone of said enarthrosis, and further comprising at least one screw for securing said side plate onto said long bone.

12. A prosthetic device as claimed in claim 10, wherein said securement means comprises a suitable intramedullary nail arrangement comprising an intramedullary nail adapted for securement thereof within a shaft formed in a medullary canal, and mounting means for reversibly mounting said ball prosthesis portion to said intramedullary nail.

13. A prosthetic device as claimed in claim 10, wherein said securement means comprises a suitable telescopic strut arrangement comprising an extendable telescopic strut adapted for securement thereof within a shaft formed in a medullary canal, and mounting means for reversibly mounting said ball prosthesis portion to said telescopic strut arrangement.

14. A prosthetic device as claimed in claim 13, wherein said telescopic strut comprises in its retracted configuration a transverse profile adapted for enabling said ball prosthesis to be implanted in said enarthrosis via said distal portal.

15. A prosthetic device as claimed in claim 13, wherein said telescopic strut comprises an outermost member integrally joined to said distal end of said body member.

16. A prosthetic device as claimed in any one of claims 1 to 15, further comprising a socket prosthesis portion having a proximal end adapted for securement onto the socket of said enarthrosis and a distal side having a concavity adapted for receiving said ball member such as to permit relative rotational movement therebetween.

17. A prosthetic device as claimed in claim 16, wherein said concavity of said socket prosthesis portion has a curvature complementary to the curvature of said ball portion.

18. A prosthetic device as claimed in claim 16, wherein said socket prosthesis portion is made from any suitable material including silicone, polyethylene, polyurethane or ceramic material.

19. A prosthetic device as claimed in claims 1 to 15, adapted for the shoulder joint, wherein said extremity is the corresponding arm.

20. A prosthetic device as claimed in claim 16, adapted for the shoulder joint, wherein said extremity is the corresponding arm.

21. A prosthetic device as claimed in claims 1 to 15, adapted for the hip joint, wherein said extremity is the corresponding leg.

22. A prosthetic device as claimed in claim 16, adapted for tile hip joint, wherein said extremity is the corresponding leg.

23. A method for installing a prosthetic device for the hip joint comprising the steps:—
(a) Forming a portal through the lateral cortex of the femur opposite the head of the femur and removing the head of the femur;
(b) Providing a prosthetic device as claimed in any one of claims 16 to 18;
(c) Preparing the acetabulum of the hip to receive said socket prosthesis portion and fixing same in place;
(d) Inserting the ball prosthesis portion through the portal and securing said body portion thereof in said portal;
(e) Closure of fascia, subcutis and skin.

24. A method for installing a prosthetic device as claimed in claim 23, wherein said prosthetic device comprises securement means as claimed in claim 10, said method further comprising the steps:—
(f) Preparing infrastructure in the femur for the securement means of the ball prosthesis portion;
(g) Securing said ball prosthesis portion with respect to the femur via said securement means.

25. A method for installing a prosthetic device as claimed in claim 24, wherein said prosthetic device comprises securement means as claimed in any one of claims 10 and 11 to 15, and wherein said step (f) is executed before step (d) and step (g) is executed after step (d).

26. A method for installing a prosthetic device as claimed in claim 24, wherein said prosthetic device comprises securement means as claimed in claim 11, and wherein steps (f) and (g) are executed after step (d).

27. A method for installing a prosthetic device for the shoulder joint comprising the steps:—
(h) Forming a portal through the lateral cortex of the humerus opposite the head of the humerus and removing the head of the humerus;
(i) Providing a prosthetic device as claimed in any one of claims 1 to 15;
(j) Inserting the ball prosthesis portion through the portal and securing said body portion thereof in said portal;
(k) Closure of fascia, subcutis and skin.

28. A method for installing a prosthetic device as claimed in claim 27, further comprising the following steps between steps (i) and (j):
(l) Preparing the glenoid of the shoulder to receive said socket prosthesis portion and fixing same in place;

29. A method for installing a prosthetic device as claimed in claim 28, wherein said prosthetic device comprises securement means as claimed in claim 10, said method further comprising the steps:—
(m) Preparing infrastructure in the humerus for the securement means of the ball prosthesis portion;
(n) Securing said ball prosthesis portion with respect to the humerus via said securement means;

30. A method for installing a prosthetic device as claimed in claim 29, wherein said prosthetic device comprises securement means as claimed in any one of claims 10 and 12 to 15, and wherein said step (m) is executed before step (j) and step (n) is executed after step (j).

31. A method for installing a prosthetic device as claimed in claim 29, wherein said prosthetic device comprises securement means as claimed in claim 11, and wherein steps (m) and (n) are executed after step (j).
